(19) United States
(12) Patent Application Publication
(10) Pub. No.: US 2024/0016367 A1
(43) Pub. Date: Jan. 18, 2024

(54) AUTHENTICATION METHOD FOR ENDOSCOPE, ENDOSCOPE SYSTEM, AND RECORDING MEDIUM

(71) Applicant: OLYMPUS MEDICAL SYSTEMS CORP., Tokyo (JP)

(72) Inventor: Kento IGUCHI, Tokyo (JP)

(73) Assignee: OLYMPUS MEDICAL SYSTEMS CORP., Tokyo (JP)

(21) Appl. No.: 18/219,148

(22) Filed: Jul. 7, 2023

Related U.S. Application Data
(60) Provisional application No. 63/389,019, filed on Jul. 14, 2022.

(51) Int. Cl. A61B 1/00

(52) U.S. Cl. CPC A61B 1/00059; A61B 1/00045; A61B 1/00055; A61B 1/00103

(57) ABSTRACT

An authentication method for endoscope includes acquiring a first identification information stored in a first memory of the endoscope, acquiring a second identification information stored in a second memory of the endoscope, comparing the acquired first identification information with the acquired second identification information, and based on a result of the comparing, performing a first processing or a second processing.

### RELATED APPLICATION DATA

This application is based on and claims priority under 35 U.S.C. § 119 to U.S. Provisional Application No. 63/389,019 filed on Jul. 14, 2022, the entire contents of which are incorporated herein by reference.

### FIELD OF DISCLOSURE

The present disclosure relates to an authentication method for endoscope, an endoscope, an endoscope observing apparatus, and a recording medium.

### BACKGROUND

Conventionally, endoscopes are widely used in medical fields, industrial fields, and the like. Generally, an endoscope is configured to include an insertion portion with an elongated tube shape and a distal end portion provided at a distal end of the insertion portion.

As such an endoscope, a so-called single-use endoscope in a form to be discarded after only one use is known. For example, as disclosed in International Publication No. 2020-110898, a single-use endoscope is configured by combining reusable components which can be repeatedly used with non-reusable components which can only be used once.

### SUMMARY

An authentication method for endoscope according to an aspect of the present disclosure includes acquiring a first identification information stored in a first memory of the endoscope, acquiring a second identification information stored in a second memory of the endoscope, comparing the acquired first identification information with the acquired second identification information, and based on a result of the comparing, performing a first processing or a second processing.

In addition, an endoscope according to an aspect of the present disclosure includes a first memory configured to store a first identification information, and a second memory configured to store a second identification information. The first identification information matches the second identification information.

Furthermore, an endoscope observing apparatus according to an aspect of the present disclosure includes a processor. The processor is configured to acquire a first identification information stored in a first memory of an endoscope, acquire a second identification information stored in a second memory of the endoscope, perform a comparison processing including comparing the acquired first identification information with the acquired second identification information, and perform a next processing based on a result of the comparison processing.

Moreover, a non-transitory recording medium on which a program is recorded according to an aspect of the present disclosure causes a computer to execute a process including acquiring a first identification information stored in a first memory of an endoscope, acquiring a second identification information stored in a second memory of the endoscope, comparison the acquired first identification information with the acquired second identification information, perform a next processing based on a result of the comparison.

### BRIEF DESCRIPTION OF THE DRAWINGS

FIG. 1 is an overall configuration diagram showing an example of an overall configuration of an endoscope system according to an embodiment.

FIG. 2 is a block diagram showing an example of a configuration of a principal part of the endoscope system.

FIG. 3 is a flow chart for explaining an example of authentication processing for endoscope.

FIG. 4 is a flow chart for explaining an example of authentication processing for endoscope according to a first modification.

FIG. 5 is a block diagram showing an example of a configuration of a principal part of an endoscope system according to a second modification.

### DETAILED DESCRIPTION

Generally, there are third parties who take out reusable components that are repeatedly reusable from a single-use endoscope after one use to remanufacture an endoscope. An endoscope remanufactured by an inappropriate third party with malicious intent may possibly cause a defect due to inadequate cleaning, sterilization, testing, or the like.

According to an embodiment described below, in a single-use endoscope, an authentication method for endoscope, an endoscope, an endoscope observing apparatus, and a recording medium which are capable of preventing fraudulent reuse can be provided.

Hereinafter, the embodiment will be described in detail with reference to the drawings.

Note that the drawings based on the embodiment are schematic in nature and that a relationship between a thickness and a width of each portion, a ratio among thicknesses and relative angles of respective portions, and the like differ from reality. Furthermore, even among the drawings, the drawings include portions having a relationship or a ratio among dimensions that differ from each other.

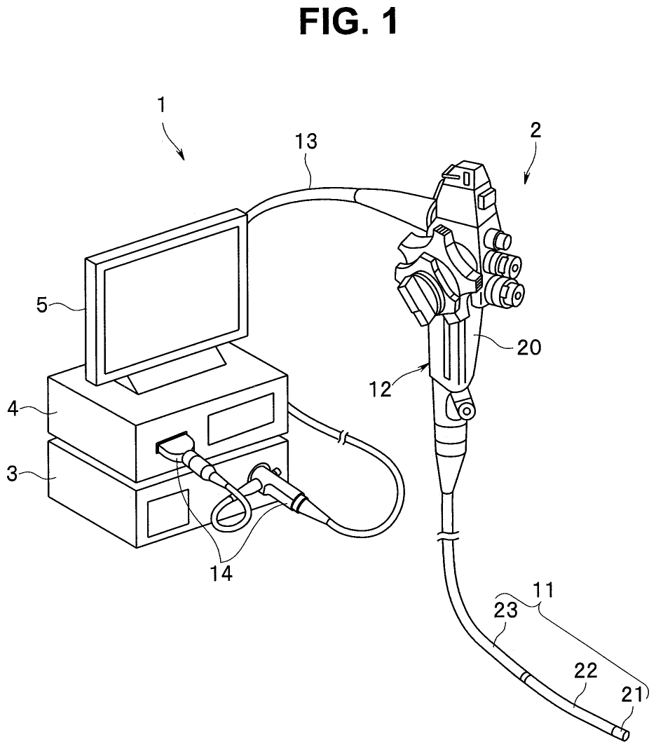

FIG. 1 is an overall configuration diagram showing an example of an overall configuration of an endoscope system according to the embodiment.

As shown in FIG. 1, a main part of an endoscope system 1 is configured to include an endoscope 2, a light source apparatus 3, a video processor 4, and a display apparatus 5 (display). The endoscope 2 is a single-use endoscope in which at least some components are used once and at least some components are used a plurality of times.

The endoscope 2 is configured to include an elongated insertion portion 11 to be inserted into an observation object site of a subject, an operating unit 12 provided continuously with a proximal end portion of the insertion portion 11, a universal cable 13 which extends from a side surface of the operating unit 12, and a connector portion 14 provided at an extended end portion of the universal cable 13. The connector portion 14 is configured to include a light source connector, an electric cable which extends from a side portion of the light source connector, and an electric connector arranged on an extended end of the electric cable. The light source connector of the connector portion 14 is removably connected to the light source apparatus 3. In addition, the electric connector of the connector portion 14 is removably connected to the video processor 4.

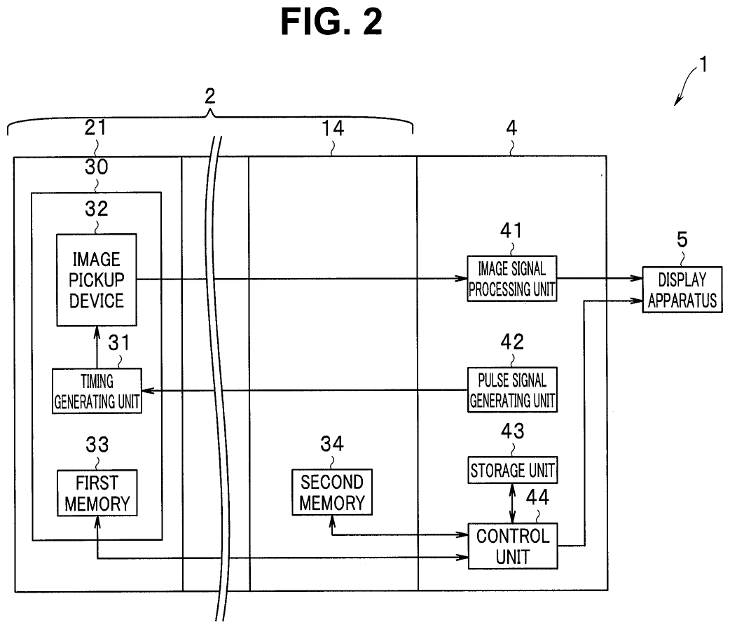

The insertion portion 11 includes a distal end portion 21 on a distal end side and a bending portion 22 which is bendable is provided continuously with a proximal end portion of the distal end portion 21. Furthermore, a flexible tube portion 23 which is elongated and flexible and which is formed of a soft tubular member is provided continuously with a proximal end portion of the bending portion 22. An image pickup unit 30 (refer to FIG. 2) for acquiring image information of the subject is provided in the distal end portion 21.

The operating unit 12 is configured to include an operating unit main body 20 which constitutes an operating grasping portion. The operating unit main body 20 is turnably provided with an angle knob for bending and operating the bending portion 22 of the insertion portion 11 and also provided with a suction button, an air/water feeding button, various switches related to various endoscope functions, and the like.

The light source apparatus 3 supplies a light guide (not illustrated) provided inside the endoscope 2 with illuminating light. Specifically, the light guide is arranged inside the universal cable 13, the operating unit 12, and the insertion portion 11 of the endoscope 2 according to the present embodiment and the light source apparatus 3 supplies an illuminating optical system which constitutes an illuminating window of the distal end portion 21 with illuminating light via the light guide. The illuminating light is diffused by the illuminating optical system and illuminates a site to be examined.

The video processor 4 applies image processing to image data picked up by the endoscope 2 to generate an image signal and outputs the generated image signal to the display apparatus 5. The display apparatus 5 displays an image corresponding to the image signal generated by the video processor 4.

In addition, the video processor 4 controls an entirety of the endoscope system 1. For example, the video processor 4 performs control for switching among types of illuminating light emitted by the light source apparatus 3 and switching among image pickup modes of the endoscope 2. Furthermore, as will be described later, the video processor 4 performs authentication processing for determining whether or not the endoscope 2 has been fraudulently remanufactured.

FIG. 2 is a block diagram showing an example of a configuration of a principal part of the endoscope system. With reference to FIG. 2, details of each component of the endoscope system 1 and a path of an electrical signal inside the endoscope system 1 will be described.

As shown in FIG. 2, the distal end portion 21 of the endoscope 2 is equipped with the image pickup unit 30. The image pickup unit 30 includes a timing generating unit 31, an image pickup device 32, and a first memory 33. In addition, the connector portion 14 of the endoscope 2 includes a second memory 34. The endoscope 2 is connected to the video processor 4 as an endoscope observing apparatus via the connector portion 14. The endoscope 2 includes the first memory 33 configured to store a first identification information, and the second memory 34 configured to store a second identification information. The first identification information matches the second identification information. The endoscope can include a single-use component and a multi-use component. The single-use component is disposed or returned to manufacturer of after being used once, and the multi-use component is a reusable component that is repeatedly used. The first memory can be provided in the multi-use component. The second memory can be provided in the single-use component.

The video processor 4 includes an image signal processing unit 41, a pulse signal generating unit 42, a storage unit 43, and a control unit 44 (processor).

The timing generating unit 31 generates a drive signal including a horizontal synchronizing signal and a vertical synchronizing signal based on a reference clock signal inputted from the pulse signal generating unit 42 and outputs the drive signal to the image pickup device 32.

The image pickup device 32 is an image sensor such as a CCD, a CMOS, or the like and picks up an optical image formed by an objective optical system based on the drive signal generated by the timing generating unit 31 and outputs an image pickup signal to the image signal processing unit 41 of the video processor 4.

The first memory 33 is a memory in which data is rewritable. The first memory 33 is constituted of, for example, a flash memory or an EEPROM and stores the first identification information. The first memory 33 is provided in the image pickup unit 30 which is a component to be used a plurality of times. Note that although the first memory 33 is provided in the image pickup unit 30, the first memory 33 is not limited to being provided in the image pickup unit 30 and may be provided in another component to be used a plurality of times such as a substrate or the like arranged inside the operating unit 12. A sensor such as a temperature sensor or an inertial sensor may be provided, in which case the first memory 33 may be provided on a circuit to which the sensor is connected. The endoscope system 1 includes the endoscope 2, and the sensor configured to monitor a subject, the operating unit 12, the universal cable 13 extends from the side surface of the operating unit 12, and the connector 14 configured to connect the universal cable to the endoscope observing apparatus 4. The first memory 33 can be located in the sensor, and the second memory 34 can be located in the connector 14. The sensor can be the image sensor 32.

On the other hand, the second memory 34 is a memory in which data is not rewritable. The second memory 34 can be, for example, a mask ROM or a one-time programmable ROM and stores the second identification information. The second memory 34 is provided in the connector portion 14 which is a component to be used once. The first identification information and the second identification information need only to enable the control unit 44 to identify each of the individual components of the endoscope 2 which incorporates such components and are, for example, a combination of alphanumeric characters of a plurality of digits.

When manufacturing the endoscope 2, the first identification information stored in the first memory 33 and the second identification information stored in the second memory 34 are the same information. In addition, preferably, the first identification information and the second identification information are respectively encrypted and stored in the first memory 33 and the second memory 34.

The image signal processing unit 41 performs predetermined image processing such as white balance adjustment processing, gain adjustment processing, and gamma correction processing with respect to the image pickup signal acquired by the image pickup device 32. The image signal processing unit 41 outputs an image signal generated by the predetermined image processing to the display apparatus 5.

The pulse signal generating unit 42 generates a reference clock signal to be a reference of operation of each constituent unit of the image pickup unit 30 and outputs the reference clock signal to the timing generating unit 31.

The storage unit 43 stores a program for executing various functions related to the endoscope system 1. In addition, the storage unit 43 stores various kinds of information related to the endoscope system 1, data being processed, and the like.

The control unit 44 is configured as a processor including hardware. For example, the control unit 44 is configured as an ASIC (application specific integrated circuit) including a CPU (central processing unit) or the like or an FPGA (field programmable gate array). The control unit 44 fulfills various functions by reading a processing program from the storage unit 43 and executing the processing program. However, at least a part of the control unit 44 may be configured as a dedicated electronic circuit.

The control unit 44 comprehensively controls each unit which constitutes the endoscope system 1. When the endoscope 2 is connected to the video processor 4, the control unit 44 reads the first identification information from the first memory 33. When the first identification information is not stored in the first memory 33, the control unit 44 prohibits the use of the endoscope 2.

When the first identification information is stored in the first memory 33, the control unit 44 reads the second identification information from the second memory 34. Next, the control unit 44 compares the first identification information and the second identification information with each other (comparison processing) and determines whether or not the pieces of identification information match each other. When the first identification information and the second identification information do not match each other, the control unit 44 prohibits the use of the endoscope 2.

As described above, when the first identification information is not stored in the first memory 33 or when the first identification information and the second identification information do not match each other, the control unit 44 performs first processing of warning of the use of the endoscope 2. More specifically, the control unit 44 causes the display apparatus 5 to display a warning message or causes a warning sound to be emitted in the video processor 4 or the display apparatus 5. The first identification information stored in the first memory 33 of the endoscope 2 is acquired (S1). The second identification information stored in the second memory 34 of the endoscope 2 is acquired (S2). The acquired first identification information is compared with the acquired second identification information (S4). Based on a result of the comparing, a first processing or a second processing can be performed. When the result of the comparing is the acquired first identification information does not match the acquired second identification information, the first processing can be performed. The first processing can include generating a warning not to use the endoscope. The generated warning can include the warning message and the first processing includes causing the display 5 to display the warning message. The endoscope can include the processor 44. The processor is configured to acquire the first identification information stored in the first memory 33 of the endoscope 2, acquire the second identification information stored in the second memory 34 of the endoscope 2, compare the acquired first identification information with the acquired second identification information acquired, and based on the result of the comparing, perform the first processing or the second processing.

On the other hand, when the first identification information and the second identification information match each other, the control unit 44 permits the use of the endoscope 2. After reading the second identification information from the second memory 34, the control unit 44 performs second processing of changing the first identification information stored in the first memory 33. More specifically, the control unit 44 performs processing of erasing the first identification information stored in the first memory 33 or rewriting the first identification information to another piece of information (or adding another piece of information to the first identification information). When the result of the comparing is the acquired first identification information matches the acquired second identification information, the second processing is performed. The second processing can include changing the first identification information stored in the first memory 33. The second processing can include deleting the first identification information stored in the first memory 33. The second processing can include adding information to the first identification information stored in the first memory 33. When the acquired first identification information does not match the acquired second identification information, the first processing can be performed. When the acquired first identification information matches the acquired second identification information, the second processing can be performed. The first processing can include generating the warning not to use the endoscope 2, and the second processing can include changing the first identification information stored in the first memory 33.

In this manner, when the endoscope 2 is used once, the first identification information in the first memory 33 which is arranged in the image pickup unit 30 is changed. When an inappropriate third party with malicious intent takes out the image pickup unit 30 from the endoscope 2 having been discarded after one use and remanufactures an endoscope, a state is created where the first identification information in the first memory 33 is erased or the first identification information and the second identification information do not match each other.

Therefore, when a fraudulently remanufactured endoscope is connected to the video processor 4, a state is created where the first identification information cannot be read from the first memory 33 or the first identification information and the second identification information do not match each other and the use of the fraudulently remanufactured endoscope can be prohibited.

At the time of remanufacturing, the first identification information can be remanufactured so that the first identification information matches the second identification information. At the time of remanufacturing, by referring to the second identification information, the first identification information that matches the second identification information can newly be stored or restored to the new or reused first memory of the new imaging unit 30, respectively. The use of the remanufactured endoscope 2 is not prohibited, because the first identification information matches the second identification information.

While the video processor 4 reads the first identification information and the second identification information from the first memory 33 and the second memory 34, respectively, of the endoscope 2 and determines whether or not the endoscope 2 has been fraudulently remanufactured in the present embodiment, the present embodiment is not limited to the configuration.

Next, operations of the endoscope system 1 configured as described above will be explained.

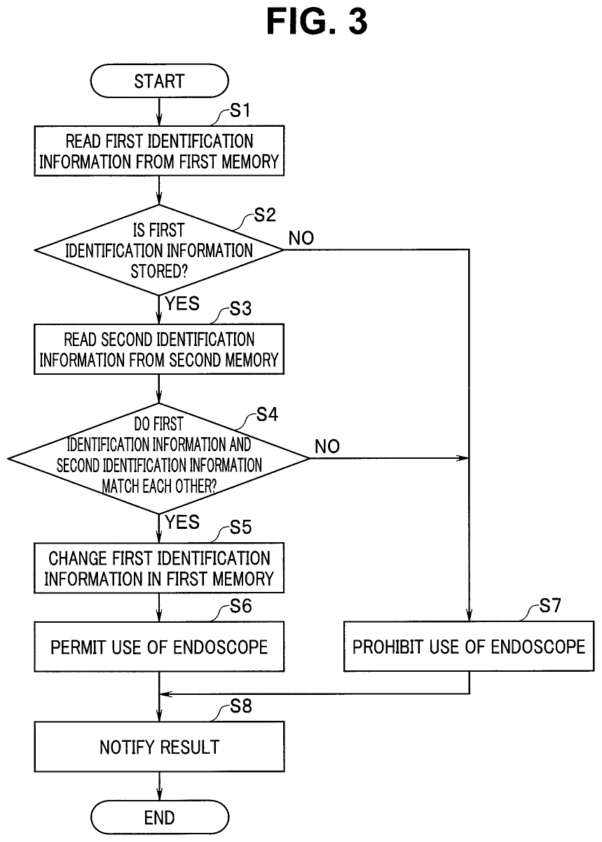

FIG. 3 is a flow chart for explaining an example of authentication processing for endoscope. The authentication processing for endoscope shown in FIG. 3 is started when the endoscope 2 is connected to the video processor 4.

First, when the endoscope 2 is connected to the video processor 4, the control unit 44 reads the first identification information from the first memory 33 (S1). Next, the control unit 44 determines whether or not the first identification information is stored in the first memory 33 (S2).

When the control unit 44 determines that the first identification information is stored in the first memory 33 (S2: YES), the control unit 44 reads the second identification information from the second memory 34 (S3). On the other hand, when the control unit 44 determines that the first identification information is not stored in the first memory 33 (S2: NO), the control unit 44 advances to the processing of S7.

Next, the control unit 44 compares the first identification information and the second identification information with each other (comparison processing) and determines whether or not the pieces of identification information match each other (S4). When the control unit 44 determines that the first identification information and the second identification information match each other (S4: YES), the control unit 44 changes the first identification information in the first memory 33 (S5). On the other hand, when the control unit 44 determines that the first identification information and the second identification information do not match each other (S4: NO), the control unit 44 advances to the processing of S7.

When the control unit 44 changes the first identification information in the first memory 33 in the processing of S5, the control unit 44 permits the use of the endoscope 2 (S6) and advances to the processing of S8. On the other hand, when the control unit 44 determines in the processing of S2 that the first identification information is not stored in the first memory 33 or determines in the processing of S4 that the first identification information and the second identification information do not match each other, the control unit 44 prohibits the use of the endoscope 2 (S7) and advances to the processing of S8.

Finally, the control unit 44 notifies a user of a result (S8) and ends the processing. For example, when the use of the endoscope 2 is permitted, the control unit 44 causes the fact that the endoscope is usable to be displayed on the display apparatus 5 or makes a transition to a standby state in which an operation from the endoscope 2 is accepted.

In addition, for example, when the use of the endoscope 2 is prohibited, the control unit 44 notifies the user that a fraudulently remanufactured endoscope is connected to the video processor 4 by causing the display apparatus 5 to display a warning message or causing a warning sound to be emitted in the video processor 4 or the display apparatus 5. Alternatively, when the use of the endoscope 2 is prohibited, the control unit 44 shuts down the video processor 4. The first processing can include generating the warning not to use the endoscope. The generated warning includes the warning sound and the first processing includes causing the warning sound to be emitted.

According to the processing described above, when the first identification information is not stored in the first memory 33 or when the first identification information and the second identification information do not match each other even if the first identification information is stored in the first memory 33, use of the endoscope 2 can be stopped. Accordingly, the endoscope system 1 can prevent an endoscope having been fraudulently remanufactured by taking out a component which can be reused a plurality of times from the single-use endoscope 2 from being fraudulently reused.

While determining whether or not the first identification information and the second identification information match each other has been heretofore described as comparison processing, comparison processing is not limited to determining whether or not the first identification information and the second identification information match each other. More specifically, by comparing the first identification information and the second identification information with each other, a determination need only be made that the first identification information and the second identification information do not form a fraudulent combination. For example, one of the first identification information and the second identification information may be an output of predetermined arithmetic processing having been performed using the other of the first identification information and the second identification information as an argument.

### (First Modification)

Next, a first modification will be described.

In the embodiment described above, after reading the second identification information from the second memory 34, the control unit 44 changes the first identification information stored in the first memory 33.

By comparison, in the first modification, the control unit 44 changes the first identification information stored in the first memory when the control unit 44 detects that the endoscope 2 and the video processor 4 have been connected for a predetermined time period or longer.

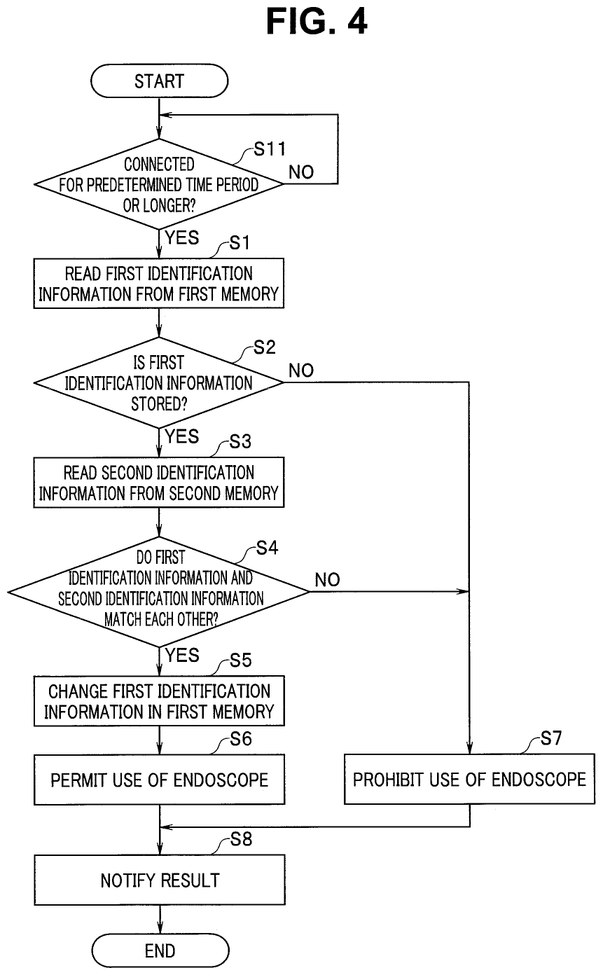

FIG. 4 is a flow chart for explaining an example of authentication processing for endoscope according to the first modification. The authentication processing for endoscope shown in FIG. 4 is started when the endoscope 2 is connected to the video processor 4. In FIG. 4, similar processing to FIG. 3 will be denoted by same reference signs and a description of such processing will be omitted.

First, when the endoscope 2 is connected to the video processor 4, the control unit 44 determines whether or not the endoscope 2 and the video processor 4 have been connected for a predetermined time period or longer (S11). When the control unit 44 determines that the endoscope 2 and the video processor 4 have not been connected for a predetermined time period or longer (S11: NO), the control unit 44 returns to the processing of S11. On the other hand, when the control unit 44 determines that the endoscope 2 and the video processor 4 have been connected for a predetermined time period or longer (S11: YES), the control unit 44 advances to the processing of S1 and reads the first identification information from the first memory 33. The processing steps of S1 and thereafter are similar to the processing steps shown in FIG. 3.

According to the processing described above, the control unit 44 changes the first identification information stored in the first memory when the endoscope 2 and the video processor 4 have been connected for a predetermined time period or longer. In other words, when the endoscope 2 and the video processor 4 have been connected for a predetermined time period or longer, the control unit 44 determines that the single-use endoscope 2 has been used once and changes the first identification information stored in the first memory 33.

As described above, since the first identification information is changed based on a connection of a predetermined time period or longer, a determination that the endoscope 2 has been used once can be prevented from being made in a case where the endoscope 2 and the video processor 4 are naturally disconnected from each other due to the connection between the endoscope 2 and the video processor 4 being insufficient or a case of a connection in a test stage prior to use of the endoscope 2.

### (Second Modification)

Next, a second modification will be described.

In the embodiment described above, the video processor 4 reads the first identification information and the second identification information from the first memory 33 and the second memory 34 of the endoscope 2 and determines whether or not the endoscope 2 has been fraudulently remanufactured.

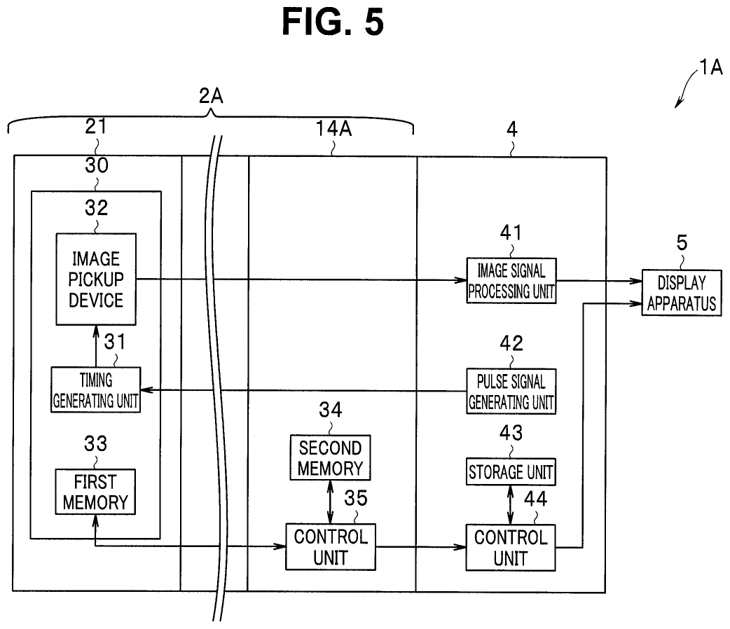

By comparison, in the second modification, a control unit including a same function as the control unit 44 of the video processor 4 is provided in an endoscope 2A (refer to FIG. 5), a control unit 35 (refer to FIG. 5) provided in the endoscope 2A reads the first identification information and the second identification information from the first memory 33 and the second memory 34, respectively, during use of the endoscope 2A and determines whether or not the endoscope 2A has been fraudulently remanufactured.

Note that “during use of the endoscope 2A” may be when power is supplied from the video processor 4 to the endoscope 2A or when a battery (not illustrated) is further incorporated in the endoscope 2A and power is supplied from the battery to each unit of the endoscope 2A.

FIG. 5 is a block diagram showing an example of a configuration of a principal part of an endoscope system according to the second modification. Note that, in FIG. 5, similar components to FIG. 2 will be denoted by same reference signs and a description of such components will be omitted.

An endoscope system 1A includes the endoscope 2A in place of the endoscope 2 shown in FIG. 2. The endoscope 2A includes a connector portion 14A in place of the connector portion 14 shown in FIG. 2. The connector portion 14A is configured by adding the control unit 35 to the connector portion 14 shown in FIG. 2.

The control unit 35 includes a similar function to the control unit 44 of the embodiment described above. More specifically, when power is supplied to the endoscope 2A, the control unit 35 reads the first identification information from the first memory 33. When the first identification information is not stored in the first memory 33, the control unit 35 prohibits the use of the endoscope 2A.

When the first identification information is stored in the first memory 33, the control unit 35 reads the second identification information from the second memory 34. Next, the control unit 35 compares the first identification information and the second identification information with each other (comparison processing) and determines whether or not the pieces of identification information match each other. When the first identification information and the second identification information do not match each other, the control unit 35 prohibits the use of the endoscope 2A.

When the first identification information is not stored in the first memory 33 or when the first identification information and the second identification information do not match each other, the control unit 35 performs first processing of warning of the use of the endoscope 2A. More specifically, the control unit 35 transmits information indicating that the first identification information is not stored in the first memory 33 or the first identification information and the second identification information do not match each other to the control unit 44 of the video processor 4 and causes the display apparatus 5 to display a warning message or causes a warning sound to be emitted in the video processor 4 or the display apparatus 5.

On the other hand, when the first identification information and the second identification information match each other, the control unit 35 permits the use of the endoscope 2A. After reading the second identification information from the second memory 34, the control unit 35 performs second processing of changing the first identification information stored in the first memory 33.

As described above, by providing the endoscope 2A with the control unit 35, the endoscope 2A can determine whether or not the endoscope 2A has been fraudulently remanufactured.

A non-transitory recording medium on which the program is recorded, the non-transitory recording medium causing a computer to execute a process including acquiring the first identification information stored in the first memory 33 of an endoscope 2, acquiring the second identification information stored in the second memory 34 of the endoscope 2, comparison the acquired first identification information with the acquired second identification information; and perform the next processing based on the result of the comparison.

Note that the respective steps in the flow charts described in the present specification may be executed by changing an order of execution, executed simultaneously in plurality, or executed in a different order for each execution unless such executions run contrary to the nature of the steps.

The present disclosure is not limited to the embodiment described above and various modifications, alterations, and the like are possible within the scope of the gist of the present disclosure.
Example 1. An authentication method for endoscope, comprising:
   acquiring first identification information included in a first memory of the endoscope;
   acquiring second identification information included in a second memory of the endoscope;
   performing comparison processing of comparing the first identification information with the second identification information; and
   performing first processing or second processing based on a result of the comparison processing.
Example 2. The authentication method for endoscope according to Example 1, wherein:
   the first processing is performed when, as the result of the comparison processing, the first identification information and the second identification information do not match each other.
Example 3. The authentication method for endoscope according to Example 2, wherein:
   the first processing warns of a use of the endoscope.
Example 4. The authentication method for endoscope according to Example 3, wherein:
   the first processing causes a display apparatus to which the endoscope is connected to display a warning message.
Example 5. The authentication method for endoscope according to Example 3, wherein:
   the first processing causes a warning sound to be emitted.
Example 6. The authentication method for endoscope according to Example 1, wherein:
   the second processing is performed when, as the result of the comparison processing, the first identification information and the second identification information match each other.
Example 7. The authentication method for endoscope according to Example 6, wherein:
   the second processing changes the first identification information.
Example 8. The authentication method for endoscope according to Example 7, wherein:
   the second processing deletes the first identification information.
Example 9. The authentication method for endoscope according to Example 7, wherein:
   the second processing adds information to the first identification information.
Example 10. An endoscope, comprising:
   a first memory configured to include first identification information; and
   a second memory configured to include second identification information.
Example 11. The endoscope according to Example 10, wherein:
   the first identification information and the second identification information match each other.
Example 12. The endoscope according to Example 10, wherein
   the first memory is provided in a sensor configured to monitor a subject, and
   the second memory is provided in a connector for connecting to an endoscope observing apparatus.
Example 13. The endoscope according to Example 12, wherein
   the sensor is an image sensor.
Example 14. The endoscope according to Example 10, wherein
   a single-use component is included in at least a part of the endoscope.
Example 15. The endoscope according to Example 14, wherein
   the first memory is provided in a component to be used a plurality of times.
Example 16. The endoscope according to Example 14, wherein
   the second memory is provided in the single-use component.
Example 17. The endoscope according to Example 16, wherein
   the second memory is a one-time programmable ROM.
Example 18. The endoscope according to Example 10, comprising
   a processor, wherein the processor is configured to:
   acquire the first identification information included in the first memory of the endoscope;
   acquire the second identification information included in the second memory of the endoscope;
   perform comparison processing of comparing the first identification information with the second identification information; and
   perform next processing based on a result of the comparison processing.
Example 19. An endoscope observing apparatus, comprising
   a processor, wherein the processor is configured to:
   acquire first identification information included in a first memory of an endoscope;
   acquire second identification information included in a second memory of the endoscope;
   perform comparison processing of comparing the first identification information with the second identification information; and
   perform next processing based on a result of the comparison processing.
Example 20. A non-transitory recording medium on which a program is recorded,
   the non-transitory recording medium causing a computer to execute:
   processing of acquiring first identification information included in a first memory of an endoscope;
   processing of acquiring second identification information included in a second memory of the endoscope;
   comparison processing of comparing the first identification information with the second identification information; and
   next processing based on a result of the comparison processing.

What is claimed is:

1. An authentication method for an endoscope, comprising:
acquiring a first identification information stored in a first memory of the endoscope;
acquiring a second identification information stored in a second memory of the endoscope;
comparing the acquired first identification information with the acquired second identification information; and
based on a result of the comparing, performing a first processing or a second processing.

2. The authentication method according to claim 1, wherein, when the result of the comparing is the acquired first identification information does not match the acquired second identification information, the first processing is performed.

3. The authentication method according to claim 2, wherein the first processing includes generating a warning not to use the endoscope.

4. The authentication method according to claim 3, wherein the generated warning includes a warning message and the first processing includes causing a display to display the warning message.

5. The authentication method according to claim 3, wherein the generated warning includes a warning sound and the first processing includes causing the warning sound to be emitted.

6. The authentication method according to claim 1, wherein, when the result of the comparing is the acquired first identification information matches the acquired second identification information, the second processing is performed.

7. The authentication method according to claim 6, wherein the second processing includes changing the first identification information stored in the first memory.

8. The authentication method according to claim 7, wherein the second processing includes deleting the first identification information stored in the first memory.

9. The authentication method according to claim 7, wherein the second processing includes adding information to the first identification information stored in the first memory.

10. The authentication method according to claim 1, wherein, when the acquired first identification information does not match the acquired second identification information, the first processing is performed,
wherein, when the acquired first identification information matches the acquired second identification information, the second processing is performed,
wherein the first processing includes generating a warning not to use the endoscope, and
wherein the second processing includes changing the first identification information stored in the first memory.

11. An endoscope, comprising:
a first memory configured to store a first identification information; and
a second memory configured to store a second identification information,
wherein the first identification information matches the second identification information.

12. An endoscope system, comprising:
an endoscope observing apparatus; and
the endoscope according to claim 11, further comprising:
a sensor configured to monitor a subject;
an operating unit;
a universal cable extends from a side surface of the operating unit; and
a connector configured to connect the universal cable to the endoscope observing apparatus,
wherein the first memory is located in the sensor, and
wherein the second memory is located in the connector.

13. The endoscope system according to claim 12, wherein the sensor is an image sensor.

14. The endoscope according to claim 11, further including a single-use component.

15. The endoscope according to claim 14, further including a multi-use component, wherein the first memory is provided in the multi-use component.

16. The endoscope according to claim 14, wherein the second memory is provided in the single-use component.

17. The endoscope according to claim 16, wherein the second memory is a one-time programmable ROM.

18. The endoscope according to claim 11, further comprising:
a processor,
wherein the processor is configured to:
acquire the first identification information stored in the first memory of the endoscope,
acquire the second identification information stored in the second memory of the endoscope,
compare the acquired first identification information with the acquired second identification information acquired, and
based on a result of the comparing, perform a first processing or a second processing.

19. A non-transitory recording medium on which a program is recorded, the non-transitory recording medium causing a computer to execute a process including:
acquiring a first identification information stored in a first memory of an endoscope;
acquiring a second identification information stored in a second memory of the endoscope;
comparison the acquired first identification information with the acquired second identification information; and
perform a next processing based on a result of the comparison.
